(19) United States
(12) Patent Application Publication
(10) Pub. No.: US 2026/0306339 A1
(43) Pub. Date: Oct. 8, 2026

(54) ACTIVE DISTURBANCE REJECTION BACKSTEPPING CONTROL SYSTEM FOR WRIST REHABILITATION ROBOT

(71) Applicant: SOUTH-CENTRAL MINZU UNIVERSITY, wuhan (CN)

(72) Inventors: Gang Li, wuhan (CN); Erhu Yang, wuhan (CN); Lu Zheng, wuhan (CN); Dongyun Zheng, wuhan (CN); Long Li, wuhan (CN); Shasha Tian, wuhan (CN); Jun Tie, wuhan (CN)

(21) Appl. No.: 19/292,937

(22) Filed: Aug. 7, 2025

(30) Foreign Application Priority Data
Apr. 1, 2025 (CN) 202510404362.5

(51) Int. Cl. A61H 1/02

(52) U.S. Cl. CPC A61H 1/0285; A61H 2201/1207

(57) ABSTRACT

An active disturbance rejection backstepping control (ADRBSC) system for a wrist rehabilitation robot is provided. The robot includes two pneumatic artificial muscle (PAM) systems as driving components. The system includes: a tracking differentiator, configured to track and differentiate a target angle signal; an extended state observer, configured to estimate a system state and a total system disturbance in real time; a nonlinear error feedback controller, configured to form an error feedback signal and generate a control torque; a backstepping controller, configured to calculate and allocate output pressures of the PAM systems; proportion-integration-differentiation controllers, configured to dynamically correct the output pressures of the PAM systems; and an adaptive combination valve, configured to execute control commands and monitor a pressure within the PAM system module. The ADRBSC system overcomes inherent problems of high nonlinearity, uncertainty, and hysteresis effects in the PAM systems, achieving high-precision trajectory tracking control of wrist joint motion.

### CROSS-REFERENCE TO RELATED APPLICATION

This application claims priority to Chinese Patent Application No. 202510404362.5, filed on Apr. 1, 2025, which is herein incorporated by reference in its entirety.

### TECHNICAL FIELD

The disclosure relates to the field of rehabilitation robot control technologies, and more particularly to an active disturbance rejection backstepping control (ADRBSC) system for a wrist rehabilitation robot.

### BACKGROUND

Wrist dysfunction is a common movement disorder, and repetitive physical training is a conventional method for restoring wrist function. However, providing long-term, repetitive physical training for a large number of individuals in need of rehabilitation requires a significant number of dedicated technical personnel. The shortage of medical resources has become a constraint on the rehabilitation process. In response to this problem, researchers have proposed a solution of robot-assisted rehabilitation. Robots can provide users with more precise and repetitive training with minimal supervision.

In response to the problem, a solution of robot-assisted rehabilitation has gradually attracted the attention of the researchers. The robots can provide patients with high-precision, repetitive training with minimal supervision, thereby reducing the workload of rehabilitation therapists while improving the efficiency and effectiveness of rehabilitation training. Over the past few decades, the researchers have developed a variety of wrist rehabilitation robot devices. These devices include single-degree-of-freedom and multi-degree-of-freedom wrist rehabilitation robots, capable of performing multiple wrist movements such as palmar flexion/dorsiflexion, radial deviation/ulnar deviation, and abduction/adduction. Most rehabilitation robots are motor-driven and have a rigid exoskeleton structure, which offers advantages such as high control precision, strong stiffness, and accurate trajectory. However, due to the rigidity of the rigid structure, when the movement exceeds the maximum tolerable angle of the patient, the rigid structure may cause secondary damage to the wrist joint, limiting its application in flexible rehabilitation.

To enhance the flexibility of rehabilitation devices, some researchers have begun to use pneumatic actuation devices, such as a pneumatic artificial muscle (PAM). Compared with traditional motor-driven systems, the PAM has advantages such as good compliance, high power-to-weight ratio, and compact structure, and are more suitable for human rehabilitation training needs. However, in practical applications, the PAM exhibits significant hysteresis, creep effects, and complex friction, which make the robot system driven by the PAM highly nonlinear, uncertain, and time-varying. These factors are intercoupled with each other, resulting in complex dynamic characteristics of the pneumatic system. Traditional linear control methods (such as proportion-integration-differentiation control, abbreviated as PID control) are unable to effectively address these complex nonlinear and uncertain problems and cannot achieve precise trajectory tracking control of wrist joint movements.

### SUMMARY

In view of the foregoing, the disclosure provides an ADRBSC system for a wrist rehabilitation robot. By combining advantages of the active disturbance rejection control and the backstepping control, a control strategy is designed that can effectively estimate and compensate for the total system disturbance. The ADRBSC system aims to overcome high nonlinearity and uncertain nonlinear issues of the PAM systems, and achieve high-precision trajectory tracking control for the wrist rehabilitation robot.

The technical solutions of the disclosure are achieved as follows. The disclosure provides the ADRBSC system for the wrist rehabilitation robot. The wrist rehabilitation robot includes two PAM systems as driving components, the two PAM systems are a first PAM system and a second PAM system, and the first PAM system and the second PAM system form a PAM system module. The ADRBSC system includes: a tracking differentiator, an extended state observer, a nonlinear error feedback controller, a backstepping controller, PID controllers, and an adaptive combination valve.

The tracking differentiator is configured to track and differentiate a target angle signal input thereinto to generate a tracking signal and a differential signal.

The extended state observer is configured to estimate a system state and a total system disturbance in real time, and output an estimated state value.

The nonlinear error feedback controller is configured to form an error feedback signal and generate a control torque based on the tracking signal, the differential signal, and the estimated state value.

The backstepping controller is configured to calculate and allocate, based on the control torque and using a dynamic model of a single-joint mechanical arm, an output pressure of the first PAM system and an output pressure of the second PAM system.

The PID controllers are configured to dynamically correct the output pressure of the first PAM system and the output pressure of the second PAM system to compensate for a hysteresis effect of proportional pressure valves.

The adaptive combination valve includes two proportional pressure valves, a control unit, and a pressure sensor, and is configured to execute control commands and monitor a pressure within the PAM system module.

On a basis of the above technical solutions, in an embodiment, the dynamic model of the single-joint mechanical arm is as follows:

$$τ = J {{θ}^{‵}}_{f} + {b}_{v} {{θ}^{‵}}_{f} + {f}_{1} = [ {F}_{1} ( {ε}_{1} , {p}_{q 1} ) {sinγ}_{1} - {F}_{2} ( {ε}_{2} , {p}_{q 2} ) {sinγ}_{2} ] r$$

where τ represents the control torque, γ₁ represents an angle between an endpoint of the first PAM system and an endpoint of a rotating frame, γ₂ represents an angle between an endpoint of the second PAM system and the endpoint of the rotating frame, J represents a rotational inertia (also referred to as moment of inertia) of a joint pulley, which can be obtained from a formula of J=½mr², m represents a weight of a handle mechanism, r is the rotational radius of the wrist joint robot. bᵥ represents a joint damping coefficient, f₁ represents an unknown external disturbance, θ_{f} represents a rotation angle of the joint pulley, p_{q1} represents a chamber pressure of the first PAM system, p_{q2} represents a chamber pressure of the second PAM system, r represents a radius of the joint pulley, F₁(ε₁,p_{q1}) represents an output pull force of the first PAM system, F₂(ε₂,p_{q2}) represents an output pull force of the second PAM system, ε₁ represents a contraction ratio of the first PAM system, ε₂ represents a contraction ratio of the second PAM system, c represents a contraction ratio of the PAM system module, ε=1−L/Lₒᵣᵢ, Lₒᵣᵢ represents an initial length of the PAM system module, and L represents an actual length of the PAM system module.

b₀=1/J, where b₀ represents a compensation coefficient, −bᵥ/J\acute{θ}_{f}+f₁/J is the total system disturbance fₛ,

$${θ}_{f 1}^{r} = {θ}_{f} ,$$

$${θ}_{f 2}^{r} = {{θ}^{‵}}_{f} ,$$

and thus a system state space formula is expressed as follows:

$$\{ \begin{matrix}{{θ}^{‵}}_{f 1}^{r} = {θ}_{f 2}^{r} \\ {{θ}^{‵}}_{f 2}^{r} = {f}_{s} + {b}_{0} τ\end{matrix}$$

$$where$$

$${θ}_{f 1}^{r}$$

represents a feedback angle, and

$${θ}_{f 2}^{r}$$

represents a feedback angular velocity, fₛ represents the total system disturbance, including the unknown external disturbance.

On a basis of the above technical solutions, in an embodiment, the tracking differentiator is designed as follows:

$$\{ \begin{matrix}{f}_{h} = fhan ( {θ}_{r 1} - {θ}_{d} , {θ}_{r 2} , {r}_{0} , {h}_{0} ) \\ {{θ}^{‵}}_{r 1} = {θ}_{r 2} \\ {{θ}^{‵}}_{r 2} = {f}_{h}\end{matrix}$$

where fhan(θᵣ₁−θ_{d},θᵣ₂,r₀,h₀) is a derivative of the feedback angular velocity θᵣ₂, and fhan(θᵣ₁−θ_{d},θᵣ₂,r₀,h₀) is specifically expressed as follows:

$$fhan ( {θ}_{r 1} - {θ}_{d} , {θ}_{r 2} , {r}_{0} , {h}_{0} ) = - {r}_{0} ( \frac{{as}_{a}}{d} + sign ( a ) - {s}_{a} sign ( a ) )$$

$$\{ \begin{matrix}d = {r}_{0} {h}_{0}^{2} , {a}_{0} = {h}_{0} {θ}_{r 2} , y = {θ}_{r 1} - {θ}_{d} + {a}_{0} \\ {a}_{1} = \sqrt{{d}^{2} + 8 d \vert y \vert} \\ {a}_{2} = {a}_{0} + sign ( y ) \frac{{a}_{1} - d}{2} \\ {s}_{y} = \frac{sign ( y + d ) - sign ( y - d )}{2} \\ a = {a}_{2} + {a}_{0} {s}_{y} + {ys}_{y} - {a}_{2} {s}_{y} \\ {s}_{a} = \frac{sign ( a + d ) - sign ( a - d )}{2}\end{matrix}$$

where r₀ represents a velocity factor of the tracking differentiator, when r₀ is larger, a tracking velocity of the tracking differentiator is faster; h₀ represents a filtering factor of the tracking differentiator, θ_{d} represents the target angle signal input into the tracking differentiator, θᵣ₁ represents the tracking signal, and θᵣ₂ represents the differential signal of θᵣ₁.

On a basis of the above technical solutions, in an embodiment, an unknown nonlinear term f(θ_{f},\dot{θ}_{f}) of the ADRBSC system is a state variable of the extended state observer, a derivative of f(θ_{f},\dot{θ}_{f}) is h(θ_{f},\dot{θ}f), i.e., h(θ_{f},\dot{θ}f)=\dot{f}(θ_{f},\dot{θ}_{f}), the extended state observer of three stages is designed as follows:

$$\{ \begin{matrix}{\tilde{e}}_{1} = {θ}_{f 1}^{g} - {θ}_{f 1}^{r} \\ {{θ}^{‵}}_{f 1}^{g} = {θ}_{f 2}^{g} - {β}_{01} {\tilde{e}}_{1} \\ {{θ}^{‵}}_{f 2}^{g} = {θ}_{f 3}^{g} - {β}_{02} fal ( {\tilde{e}}_{1} , {a}_{1} , δ ) + {b}_{0} τ \\ {{θ}^{‵}}_{f 3}^{g} = - {β}_{03} fal ( {\tilde{e}}_{1} , {a}_{1} , δ )\end{matrix}$$

where \tilde{e}₁ represents an error between an angle observed value

$${θ}_{f 1}^{g}$$

and an output angle (i.e., the feedback angle)

$${θ}_{f 1}^{r} , {θ}_{f 1}^{g}$$

represents an estimated value of

$${θ}_{f 1}^{r} , {θ}_{f 2}^{g}$$

represents an estimated value of

$${θ}_{f 2}^{r} , {θ}_{f 3}^{g}$$

represents an estimated value of

$${θ}_{f 3}^{r} , {θ}_{f 3}^{r}$$

represents an extended state, β₀₁, β₀₂ and β₀₃ represent gain coefficients of the extended state observer; and a function ƒal(\tilde{e}ᵢ,aᵢ,δ) is defined as follows:

$$fal ( {\tilde{e}}_{i} , {a}_{i} , δ ) = \{ \begin{matrix}\frac{{\tilde{e}}_{i}}{{δ}^{1 - a}} , & \vert {\tilde{e}}_{i} \vert ⩽ δ \\ {\vert {\tilde{e}}_{i} \vert}^{{a}_{i}} sign ( {\tilde{e}}_{i} ) & \vert {\tilde{e}}_{i} \vert > δ\end{matrix}$$

where \tilde{e}ᵢ represents the error between the estimated angular velocity and the feedback angular velocity, and

$${\tilde{e}}_{i} = {θ}_{fi}^{g} - {θ}_{fi}^{r} ,$$

i=1, 2, 3; δ represents a linear interval length, and aᵢ represents a feedback exponent.

On a basis of the above technical solutions, in an embodiment, the nonlinear error feedback controller is designed as follows:

$$\{ \begin{matrix}{e}_{1} = {θ}_{r 1} - {θ}_{f 1}^{g} \\ {e}_{2} = {θ}_{r 2} - {θ}_{f 2}^{g} \\ τ = \frac{{β}_{1} fal ( {e}_{1} , {a}_{i} , {δ}_{1} ) + {β}_{2} fal ( {e}_{2} , {a}_{i} , {δ}_{1} ) - {θ}_{f 3}^{g}}{{b}_{0}}\end{matrix}$$

where e₁ represents an error between a given angle θᵣ₁ and an estimated angle value

$${θ}_{f 1}^{g} ,$$

e₂ represents an error between a given angle θᵣ₂ and an estimated angle value

$${θ}_{f 2}^{g} ,$$

β₁ represents a gain coefficient used to correct the error e₁, β₂ represents a gain coefficient used to correct the error e₂, and δ₁ represents a length of a linear region of the nonlinear function.

On a basis of the above technical solutions, in an embodiment, the backstepping controller is configured to perform the following steps:
A1, based on the control torque, using an inverse operation of the dynamic model of the single-joint mechanical arm to calculate an output pull force of the first PAM system and an output pull force of the second PAM system when a wrist joint system of the PAM system module rotates by an angle θ_{d}; and
A2, substituting the output pull force of the first PAM system and the output pull force of the second PAM system into a PAM system mathematical model to calculate the output pressure of the first PAM system and the output pressure of the second PAM system.

On a basis of the above technical solutions, in an embodiment, the PAM system mathematical model is expressed as follows:

$$\{ \begin{matrix}F ( ε , {p}_{Q} ) = {g ( ε )}_{{p}_{q}} + h ( ε ) \\ g ( ε ) = 778 - 2423 ε + 2 4.16 exp ( - 3 8.74 ε ) \\ h ( ε ) = - 121.3 - 119.9 ε + 1 30.5 exp ( - 3 8.74 ε )\end{matrix}$$

where F(ε,p_{q}) represents the output pull force of the PAM system module, E represents a contraction ratio of the PAM system module, and p_{q} represents a chamber pressure of the PAM system module.

On a basis of the above technical solutions, in an embodiment, calculation formulas of the output pull force of the first PAM system and the output pull force of the second PAM system are as follows:

$$\{ \begin{matrix}{F}_{1} ( {ε}_{1} , {p}_{q 1} ) = \frac{τ}{r sin {γ}_{1}} , {F}_{2} ( {ε}_{2} , {p}_{q 2} ) = 0 & τ > 0 \\ {F}_{1} ( {ε}_{1} , {p}_{q 1} ) = 0 , {F}_{2} ( {ε}_{2} , {p}_{q 2} ) = \frac{τ}{r sin {γ}_{2}} & τ < 0 \\ {F}_{1} ( {ε}_{1} , {p}_{q 1} ) = 0 , {F}_{2} ( {ε}_{2} , {p}_{q 2} ) = 0 & τ = 0\end{matrix}$$

where F₁(ε₁,p_{q1}) represents the output pull force of the first PAM system, F₂(ε₂,p_{q2}) represents the output pull force of the second PAM system, i represents the control torque, and r represents a radius of a joint pulley.

Calculation formulas of the output pressure of the first PAM system and the output pressure of the second PAM system are as follows:

$$\{ \begin{matrix}{p}_{r 1} = \frac{{F}_{1} ( {ε}_{1} , {p}_{q 1} ) - h ( {ε}_{1} )}{g ( {ε}_{1} )} \\ {p}_{r 2} = \frac{{F}_{2} ( {ε}_{2} , {p}_{q 2} ) - h ( {ε}_{2} )}{g ( {ε}_{2} )}\end{matrix}$$

where pᵣ₁ represents the output pressure of the first PAM system, and pᵣ₂ represents the output pressure of the second PAM system.

On a basis of the above technical solutions, in an embodiment, the ADRBSC system further includes: a sine wave function generation module and a minimum acceleration model generation module.

The sine wave function generation module is configured to generate the target angle signal as follows:

$${θ}_{d} = A sin ( π ft - 0.5 π ) + B$$

where θ_{d} represents the target angle signal, A represents a sine wave amplitude, f represents a sine wave frequency, t represents a time value, and B represents a sine wave angle bias.

The minimum acceleration model generation module is configured to generate an optimal wrist motion curve as follows:

$${θ}_{d} = {θ}_{0} + ( {θ}_{1} - {θ}_{0} ) ( \frac{10 {t}^{3}}{{d}^{3}} - \frac{15 {t}^{4}}{{d}^{4}} + \frac{6 {t}^{5}}{{d}^{5}} ) , d = {τ}_{1} - {τ}_{0}$$

$${θ}_{0} = 10 , {θ}_{1} = 10 when t ∈ [ nT , 1 / 4 T + nT )$$

$${θ}_{0} = 10 , {θ}_{1} = 50 when t ∈ [ 1 / 4 T + T , 1 / 2 T + nT )$$

$${θ}_{0} = 50 , {θ}_{1} = 50 when t ∈ [ 1 / 2 T + T , 3 / 4 T + nT )$$

$${θ}_{0} = 50 , {θ}_{1} = 10 when t ∈ [ nT , 1 / 4 T + nT )$$

where θ₀ represents an original position of a joint movement, θ₁ represents a stopping position of the joint movement, T represents a period of an optimal wrist motion control curve, n represents a number of repetitions of a wrist motion control curve, t represents a time value, d represents an interval time, t₁ represents a stopping moment of the joint movement, and t₀ represents a starting moment of the joint movement.

On a basis of the above technical solutions, in an embodiment, the ADRBSC system is configured to perform the following steps:
S1, receiving, by the tracking differentiator, the target angle signal, and generating, by the tracking differentiator, the tracking signal which is smooth and the differential signal of the tracking signal;
S2, estimating, by the extended state observer, the system state and the total system disturbance according to a system input and a system output;
S3, generating, by the nonlinear error feedback controller, the control torque based on the system state and the total system disturbance;
S4, allocating, by the backstepping controller, the output pressure of the first PAM system and the output pressure of the second PAM system according to system dynamics and a PAM system mathematical model;
S5, tracking and controlling, by the PID controllers, the output pressure of the first PAM system and the output pressure of the second PAM system;
S6, executing, by the adaptive combination valve, the control commands, and adjusting, by the adaptive combination valve, the pressure within the PAM system module; and
S7, recording, by an encoder, a rotation angle of a wrist joint in real time, and feeding, by the encoder, the rotation angle of the wrist joint back to the ADRBSC system to form a closed-loop control.

In an embodiment, each of the sine wave function generation module, the minimum acceleration model generation module, the control unit, the PAM system mathematical model, and the dynamic model of the single-joint mechanical arm is embodied by at least one processor and at least one memory coupled to the at least one processor, and the at least one memory stores computer programs executable by the at least one processor. In one embodiment, each of the sine wave function generation module, the minimum acceleration model generation module, the PAM system mathematical model, and the dynamic model of the single-joint robotic arm is a mathematical model program within the control board. The control unit is the adaptive combination valve control board, which generates control signals based on the calculation results of these programs, driving the entire system (such as the robotic arm, the PAM system, etc.) to perform the corresponding actions.

Compared to the related art, the ADRBSC system for the wrist rehabilitation robot of the disclosure has the following beneficial effects.
(1) The disclosure proposes a wrist rehabilitation robot control system that combines the active disturbance rejection control with the backstepping control. The PAM systems are used as the driving components. Through the collaborative operation of modules such as the tracking differentiator, the extended state observer, the nonlinear error feedback controller, the backstepping controller, and the PID controllers, the ADRBSC system can achieve high-precision trajectory tracking control of the wrist joint movement, and provide the adaptability of flexible drive and the safety of rehabilitation training, thereby enhancing the efficiency and effectiveness of wrist joint rehabilitation training, and providing reliable technical support for the field of wrist rehabilitation robots.
(2) By using the tracking differentiator to smooth the input target angle signal, the smooth tracking signal and its differential signal are generated, high-frequency noise is eliminated from the input signal, and the dynamic response performance is optimized. The tracking differentiator provides a high-quality input signal for subsequent controllers, avoiding control instability caused by signal fluctuations, thereby ensuring the system stability and accuracy.
(3) By using the extended state observer to estimate the system state and the total disturbance in real time, accurate state information and disturbance compensation capabilities are provided for the ADRBSC system. The core is to treat an unknown disturbance as the extended state and perform real-time observations, effectively addressing the uncertainty issues caused by the external disturbance and the internal nonlinearities in the PAM systems, and enhancing the interference rejection capability and control precision of the system.
(4) The nonlinear error feedback mechanism enables rapid response to changes in the input signal, suppresses the accumulation of errors, and ensures high-precision control of the system in complex environments, meeting the high demands for trajectory tracking in wrist joint rehabilitation training.
(5) By integrating the backstepping controller with the dynamic model of the single-joint mechanical arm, the complex nonlinear control problem is decomposed into multiple sub-problems through a step-by-step approach, and the output pressure of the first PAM system and the output pressure of the second PAM system are accurately calculated, ensuring the smoothness and precision of the wrist joint movement by properly allocating the driving forces, while also enhancing the dynamic response capability of the system.
(6) By using the PID controllers to dynamically adjust the output pressure of the PAM system module, the hysteresis effect of the proportional pressure valves is compensated, and the pressure control parameters are adjusted in real time, improving the precision and robustness of the pressure control, and ensuring the stability of the drive system during rehabilitation training.
(7) The adaptive combination valve monitors and regulates the gas pressure inside the PAM system module in real time, ensuring flexible driving characteristics of the PAM systems, and meeting the requirements for safety and adaptability in rehabilitation training.

### BRIEF DESCRIPTION OF DRAWINGS

In order to provide a clearer explanation of embodiments of the disclosure or the technical solutions in the related art, a brief introduction will be given to the accompanying drawings required for the description of the embodiments or the prior art. It is apparent that the accompanying drawings described below are only some embodiments of the disclosure. For those skilled in the art, other drawings can be obtained based on these drawings without creative labor.

FIG. 1 illustrates a schematic diagram of a system structure of a wrist rehabilitation robot of the disclosure.

FIG. 2A and FIG. 2B respectively illustrate schematic diagrams of a PAM wrist joint device and a simplified device structure of the disclosure.

FIG. 3 illustrates a schematic structural diagram of an ADRBSC system of the disclosure.

FIG. 4 illustrates an experimental result diagram of a torque generated by an active disturbance rejection control algorithm for sine wave trajectory tracking control of the disclosure.

FIG. 5 illustrates an experimental result diagram of a pressure allocation of a first PAM system by a backstepping method for the sine wave trajectory tracking control of the disclosure.

FIG. 6 illustrates an experimental result diagram of a pressure allocation of a second PAM system by the backstepping method for the sine wave trajectory tracking control of the disclosure.

FIG. 7 illustrates an experimental result diagram of ADRBSC sine wave trajectory tracking control of the disclosure.

FIG. 8 illustrates an experimental result diagram of a torque generated by an active disturbance rejection control algorithm for optimal wrist curve trajectory control of the disclosure.

FIG. 9 illustrates an experimental result diagram of a pressure allocation of the first PAM system by the backstepping method for the optimal wrist curve trajectory tracking control of the disclosure.

FIG. 10 illustrates an experimental result diagram of a pressure allocation of the second PAM system by the backstepping method for the optimal wrist curve trajectory tracking control of the disclosure.

FIG. 11 illustrates an experimental result diagram of ADRBSC optimal wrist curve trajectory tracking control of the disclosure.

### DETAILED DESCRIPTION OF EMBODIMENTS

The technical solutions in the embodiments of the disclosure will be clearly and completely described below in conjunction with the embodiments of the disclosure. Apparently, the described embodiments are only a part of the embodiments of the disclosure, not all of them.

Based on the embodiments of the disclosure, all other embodiments obtained by those skilled in the art without creative labor are within the scope of protection of the disclosure.

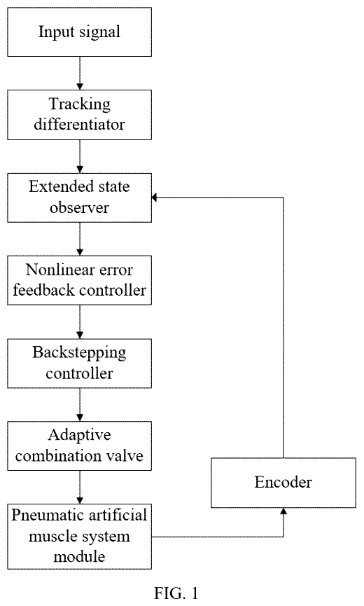

As shown in FIG. 1, the disclosure provides an ADRBSC system for a wrist rehabilitation robot. The wrist rehabilitation robot includes two PAM systems as driving components, the two PAM systems are a first PAM system and a second PAM system, and the first PAM system and the second PAM system form a PAM system module. The ADRBSC system includes: a tracking differentiator, an extended state observer, a nonlinear error feedback controller, a backstepping controller, PID controllers and an adaptive combination valve.

The tracking differentiator is configured to track and differentiate a target angle signal input thereinto to generate a tracking signal and a differential signal.

The extended state observer is configured to estimate a system state and a total system disturbance in real time, and output an estimated state value.

The nonlinear error feedback controller is configured to form an error feedback signal and generate a control torque based on the tracking signal, the differential signal, and the estimated state value.

The backstepping controller is configured to calculate and allocate, based on the control torque and using a dynamic model of a single-joint mechanical arm, an output pressure of the first PAM system and an output pressure of the second PAM system.

The PID controllers are configured to track and control the output pressure of the first PAM system and the output pressure of the second PAM system respectively to compensate for a hysteresis effect of proportional pressure valves.

The adaptive combination valve includes two proportional pressure valves, a control unit and a pressure sensor, and is configured to execute control commands and monitor a pressure within the PAM system module.

Specifically, the ADRBSC system for the wrist rehabilitation robot provided by the disclosure, by combining advantages of the active disturbance rejection control and the backstepping control, overcomes high nonlinearity and uncertainty issues of the PAM systems, and achieves high-precision trajectory tracking control for the wrist joint movement. The ADRBSC system uses the tracking differentiator to generate the smooth input signal, the extended state observer to estimate the system state and the total disturbance in real time, the nonlinear error feedback mechanism to generate the accurate control torque, the backstepping controller to allocate the output pressure of the PAM system module reasonably based on the dynamic model, the PID controllers to dynamically correct the pressure to compensate for the hysteresis effect of the proportional pressure valves, and the adaptive combination valve to ensure the accuracy and real-time performance of the pressure regulation, thereby achieving high efficiency, stability, and safety of the flexible drive, significantly enhancing the effectiveness of rehabilitation training and the adaptability of the system.

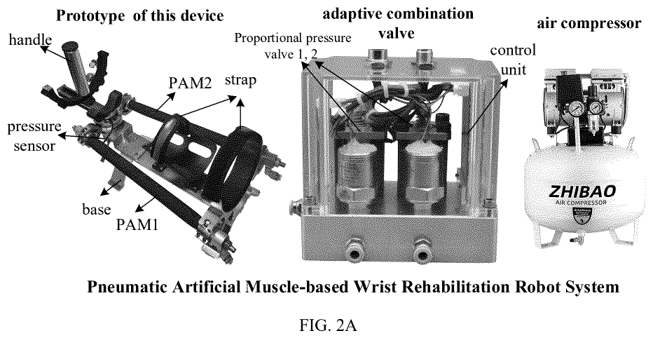

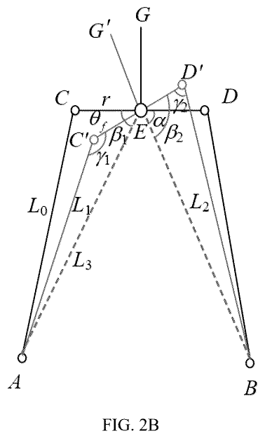

Specifically, the wrist rehabilitation robot of the disclosure controls one degree of freedom of a joint with two antagonistic PAMs (i.e., the two PAM systems) to move a human wrist for palmar flexion/dorsiflexion movements, thereby performing rehabilitation tasks. As shown in FIG. 2A, the wrist rehabilitation robot of the disclosure includes the two PAM systems as the driving components, and the robot body mainly includes a base, a wrist joint support frame, a rotating shaft, the PAM systems, a displacement sensor, a pull force sensor, and the adaptive combination valve. The base is configured to provide stable support for the entire device, and fix the wrist joint support frame and other components. The wrist joint support frame is configured to secure the wrist of a patient, and provide support and restraint to ensure the stability of the wrist during rehabilitation training. The rotating shaft is configured as a center of the wrist joint motion to connect the PAM systems to the wrist joint support frame, and convert the pull forces of the PAM systems into the rotational movement of the wrist joint; the PAM systems are configured as the driving components to generate pull forces by inflating and deflating, and drive the wrist joint to perform the palmar flexion and dorsiflexion movements. The displacement sensor is connected to the rotating shaft to detect the rotation angle and the displacement change of the wrist joint in real time, and provide feedback data of the motion states. The pull force sensor is installed at the connection position between the PAM systems and the rotating shaft to detect the pull forces generated by the PAM systems, and monitor the force conditions during wrist joint movement to thereby ensure the safety of the rehabilitation training. The adaptive combination valve includes a first proportional pressure valve, a second proportional pressure valve, the control unit, and the pressure sensor. The inlet of the first proportional pressure valve is connected to an air compressor, and the outlet of the first proportional pressure valve is connected to the PAM systems to inflate the PAM systems.

The inlet of the second proportional pressure valve is connected to the PAM systems, and the outlet of the second proportional pressure valve is connected to the atmosphere to release the internal air of the PAM systems. The pressure sensor is configured to monitor the internal pressure of the PAM systems in real-time, and transmit data to the control unit. The control unit is configured to adjust the opening of the proportional pressure valves according to the output signal of the ADRBSC system, and control the flow rate of the inflation and deflation of the PAM systems.

Under no-load conditions and when the PAM systems are not inflated, the wrist joint is in a neutral position, and the lengths of the PAM systems are their initial lengths. When the palmar flexion or dorsiflexion movement is required, the ADRBSC system inflates one of the PAM systems at one side (such as the first PAM), increase the internal pressure of the first PAM to cause contraction and generate a pull force; at the same time, another of the PAM systems on the other side (such as the second PAM) is deflated, increasing its length. The pull force of the PAM systems is transmitted to the wrist joint support frame through the rotating shaft, driving the wrist joint to move around the rotating shaft. The displacement sensor and the pull force sensor can respectively monitor and detect the displacement and the pull force in real time.

Specifically, as shown in FIG. 2B, the first PAM and the second PAM are installed on two sides of the wrist joint in an antagonistic manner, and are responsible for the forward (palmar flexion) and backward (dorsiflexion) movements of the wrist joint respectively. With no load and an inflation pressure of 0, the initial length of the PAM system module is Lₒᵣᵢ. Under a certain load, the PAM systems at the two sides are inflated to a pressure P₀, at this point the length of each PAM system is L₀, and L₀ is determined when installing the PAM structure. AC=BD=L₀, AE=BE=L₃, CE=r, ∠BED=α. When CD rotates by an angle ∠CEC′=6O, AC′=L₁, L₁=L₀−ΔL₁, BD′=L₂, L₂=L₀+ΔL₂; where ΔL₁ represents a contraction amount of the PAM system, and ΔL₂ represents an extension amount of the PAM system; ∠AEC′=β₁, ∠BED′=2, ∠AC′ε=γ₁, ∠BD′ε=γ₂, and it can be obtained by the cosine theorem as follows:

$$α = arc cos \frac{( {L}_{3}^{2} + {r}^{2} - {L}_{0}^{2} )}{2 {L}_{3} r} \qquad ( 1 )$$

Based on geometric relationships, β₁ and β₂ can be obtained as follows:

$$\{ \begin{matrix}{β}_{1} = α - {θ}_{f} \\ {β}_{2} = α + {θ}_{f}\end{matrix} \qquad ( 2 )$$

A real length L₁ of the first PAM system at left and a real length L₂ of the second PAM system at right can be obtained according to the cosine theorem. Calculation formulas of L₁ and L₂ are as follows:

$$\{ \begin{matrix}{L}_{1} = \sqrt{{L}_{{3}^{2}} + {r}^{2} - 2 {L}_{3} r cos {β}_{1}} \\ {L}_{2} = \sqrt{{L}_{{3}^{2}} + {r}^{2} - 2 {L}_{3} r cos {β}_{2}}\end{matrix} \qquad ( 3 )$$

L₁ and L₂ are obtained according to the formula (3), and the contraction ratio Ei of the first PAM system and the contraction ratio ε₂ of the second PAM system are calculated based on L₁ and L₂. Calculation formulas of ε₁ and ε₂ are as follows:

$$\{ \begin{matrix}{ε}_{1} = \frac{{L}_{ori} - {L}_{1}}{{L}_{ori}} \\ {ε}_{2} = \frac{{L}_{ori} - {L}_{2}}{{L}_{ori}}\end{matrix} \qquad ( 4 )$$

The angles ∠AC′E and ∠BD′E can be obtained according to the cosine theorem based on L₁ and L₂. Calculation formulas of ∠AC′E and ∠BD′E are as follows:

$$\{ \begin{matrix}{γ}_{1} = arc cos \frac{{L}_{1}^{2} + {r}^{2} - 2 {L}_{1} r}{2 {L}_{1} r} \\ {γ}_{2} = arc cos \frac{{L}_{2}^{2} + {r}^{2} - 2 {L}_{2} r}{2 {L}_{2} r}\end{matrix} \qquad ( 5 )$$

Specifically, the PAM is a pneumatically driven flexible actuator with unique dynamic characteristics, such as hysteresis and susceptibility to thermal factors. Establishing a dynamic model of the PAM system is relatively challenging. Therefore, the disclosure designs a controller based on the existing static mathematical model of the PAM system, to directly describe, by simplifying the complex dynamic behavior, the relationship between the contraction ratio, the chamber pressure, and the output pull force of the PAM system. First, the static mathematical model of the PAM system is as follows:

$$F ( ε , {p}_{q} ) = {k}_{1} ( {p}_{q} ) - {k}_{2} ( {p}_{q} ) ε + {k}_{3} ( {p}_{q} ) exp ( - με ) \qquad ( 6 )$$

where F(ε,p_{q}) represents the output pull force of the PAM system module, E represents the contraction ratio of the PAM,

$$ε = 1 - \frac{L}{{L}_{ori}} ,$$

Lₒᵣᵢ represents the initial length of the PAM system module, L represents an actual length of the PAM system module, p_{q} represents a chamber pressure of the PAM system module, and kᵢ(p_{q}) represents a candidate function family related to p_{q}, where i=1, 2, 3; kᵢ(p_{q})=kᵢ(p_{q})+kᵢ₂, where kᵢ₁ and kᵢ₂ represent fitting coefficients, and μ represents a nonlinear attenuation coefficient of ε.

Specifically, in the static mathematical model, the output pull force of the PAM system is decomposed into three parts, where k₁(p_{q}) represents a pull force part that changes linearly with the chamber pressure, −k₂(p_{q})ε represents a compensatory part that changes with the chamber pressure, and k₃(p_{q})exp(−με) represents a nonlinear part that changes with the contraction ratio index.

By to simplifying the kᵢ(p_{q}) function via linear fitting, it is assumed that kᵢ(p_{q})=k₁₁ (p_{q})+k₁₂, k₂(p_{q})=k₂₁(p_{q})+k₂₂, and k₃(p_{q})=k₃₁(p_{q})+k₃₂, the above assumptions are substituted into the formula (6) to obtain the following formula:

$$F ( ε , {p}_{q} ) = g ( ε ) {p}_{q} + h ( ε ) = ( {k}_{11} {p}_{q} + {k}_{12} ) - ( {k}_{21} {p}_{q} + {k}_{22} ) ε + ( {k}_{31} {p}_{q} + {k}_{32} ) exp ( - με ) \qquad ( 7 )$$

where g(E)=k₁₁−k₂₁ε+k₃₁exp(−με) represents an amplification effect of the air pressure on the output pull force, and the response sensitivity of the output pull force to the air pressure is different under various contraction ratios; and h(E)=k₁₂−k₂₂+k₃₂exp(−με) represents a nonlinear force caused by the geometry and material characteristics of the PAM itself.

Furthermore, by combining experimental data to fit k₁₁, k₁₂, k₂₁, k₂₂, k₃₁, and k₃₂, calculation formulas of the PAM system mathematical model are obtained as follows:

$$\{ \begin{matrix}F ( ε , {p}_{q} ) = g ( ε ) {p}_{q} + h ( ε ) \\ g ( ε ) = 778 - 2423 ε + 2 4.16 exp ( - 3 8.74 ε ) \\ h ( ε ) = - 121.3 - 1 19.9 ε + 1 30.5 exp ( - 3 8.74 ε )\end{matrix} \qquad ( 8 )$$

where F(ε,p_{q}) represents the output pull force of the PAM system module, E represents the contraction ratio of the PAM system module, p_{q} represents the chamber pressure of the PAM system module. It should be noted that, the PAM system module includes the first PAM system and the second PAM system, the PAM system mathematical model is applicable to both the first PAM system and the second PAM system. Therefore, when calculating the first PAM system, the output pull force of the PAM system module is the output pull force of the first PAM system, the contraction ratio of the PAM system module is the contraction ratio of the first PAM system, and the chamber pressure of the PAM system module is the chamber pressure of the first PAM system. When calculating the second PAM system, the output pull force of the PAM system module is the output pull force of the second PAM system, the contraction ratio of the PAM system module is the contraction ratio of the second PAM system, and the chamber pressure of the PAM system module is the chamber pressure of the second PAM system.

Specifically, the disclosure further includes the two proportional pressure valves. A control rate of a proportional pressure valve is approximately linearly related to the controlled output pressure. The proportional pressure valve model is expressed as follows:

$$p = {k}_{1} u + {b}_{1} \qquad ( 9 )$$

where u represents the control rate of the proportional pressure valve, p represents the controlled output pressure of the proportional pressure valve, and k₁ and b₁ represent fitting coefficients.

Due to the influence of friction between the valve core and the valve seat, the proportional pressure valve will exhibit hysteresis, causing a lag or asymmetry between the output p and variation of the input u. Therefore, the coefficients k₁ and b₁ in the formula (9) are variable.

Specifically, through relevant mechanical and physical theories, the dynamic model of the single-joint mechanical arm can be expressed as follows:

$$τ = J {{θ}^{`}}_{f} + {b}_{v} {{θ}^{`}}_{f} + {f}_{1} = [ {F}_{1} ( {ε}_{1} , {p}_{q 1} ) sin {γ}_{1} - {F}_{2} ( {ε}_{2} , {p}_{q 2} ) sin {γ}_{2} ] r \qquad ( 10 )$$

where τ represents the control torque, J represents a rotational inertia of a joint pulley, which can be obtained from a formula of J=½mr², bᵥ represents a joint damping coefficient, f₁ represents an unknown external disturbance, θ_{f} represents a rotation angle of the joint pulley, P_{q1} represents the chamber pressure of the first PAM system, p_{q2} represents the chamber pressure of the second PAM system, r represents a radius of the joint pulley, F₁(ε₁,p_{q1}) represents the output pull force of the first PAM system, F₂(ε₂>p_{q2}) represents the output pull force of the second PAM system, ε represents the contraction ratio of the PAM system module, ε=1−L/Lₒᵣᵢ, Lₒᵣᵢ represents the initial length of the PAM system module, and L represents the actual length of the PAM system module.

By rearranging the formula (10), the following formula can be obtained:

$${{θ}^{`}}_{f} = \frac{τ}{J} - \frac{{b}_{v}}{J} {{θ}^{`}}_{f} + \frac{{f}_{1}}{J} \qquad ( 11 )$$

b₀=1/J, −bᵥ/J\acute{θ}_{f}+f₁/J is considered as the total system disturbance fₛ, and the formula (11) is rewritten as follows:

$${{θ}^{`}}_{f} = {b}_{0} τ + {f}_{s} \qquad ( 12 )$$

$${θ}_{f 1}^{r} = {θ}_{f} , {θ}_{f 2}^{r} = {{θ}^{`}}_{f} ,$$

the formula (12) is written in a state space form as follows:

$$\{ \begin{matrix}{{θ}^{`}}_{f 1}^{r} = {θ}_{f 2}^{r} \\ {{θ}^{`}}_{f 2}^{r} = {f}_{s} + {b}_{0} τ\end{matrix} \qquad ( 13 )$$

where fₛ represents the total system disturbance, including the unknown external disturbance.

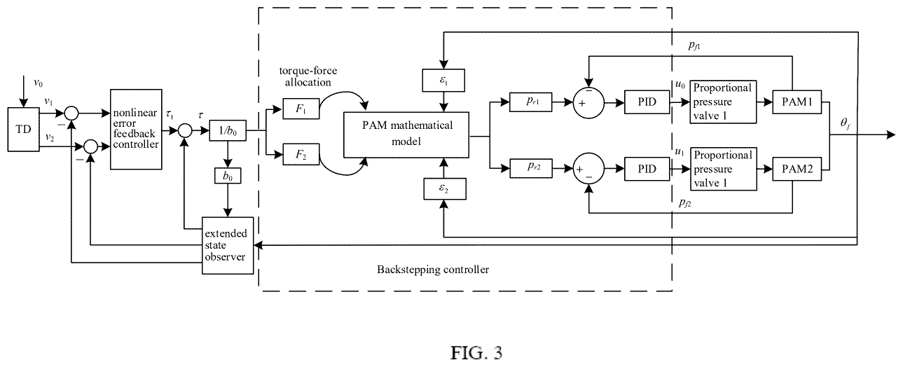

The ADRBSC system provided by the disclosure uses a controller combined with the backstepping method and the active disturbance rejection sliding mode control to achieve precise control of the joint angle. The structural diagram of the ADRBSC system is shown in FIG. 3. The ADRBSC system includes the tracking differentiator, the extended state observer, the nonlinear error feedback controller, and the backstepping controller. The working process is as follows.

The ADRBSC system receives the target angle signal v₀, and smooths, by the tracking differentiator, the target angle signal to generate a smooth input tracking signal v₁ and its differential signal v₂. The extended state observer estimates, according to the input signal and the output signal of the ADRBSC system, the system state and the total disturbance in real time to generate a compensation value b₀n, which provides accurate state information and disturbance compensation capability for subsequent control. The nonlinear error feedback controller (NLEFC) generates the control torque r based on the tracking signal v₁, the differential signal v₂ and the estimated state value. The control torque r is transformed into forces F₁ and F₂ through the PAM system mathematical model, and the forces F₁ and F₂ are allocated to the first PAM system (PAM1 in FIG. 3) and the second PAM system (PAM2 in FIG. 3) through a torque-force allocation mathematical model. The forces F₁ and F₂ are transformed into pressure commands pᵣ₁ and pᵣ₂, and the pressure commands pᵣ₁ and pᵣ₂ are sent to the two PID controllers. The pressure signals are dynamically corrected to compensate for the hysteresis effect of the proportional pressure valves (proportional pressure valve 1 and proportional pressure valve 2 in FIG. 3). The first PAM system and the second PAM system work in an antagonistic manner to control the forward (palmar flexion) and backward (dorsiflexion) movements of the wrist joint. An encoder records the actual output angle Bf of the wrist joint in real time and feeds it back to the ADRBSC system, forming a closed-loop control to ensure that the ADRBSC system dynamically adjusts the control strategy according to the actual motion state.

The tracking differentiator is a nonlinear dynamic component that effectively extracts the differential signal. The input signal is processed by the tracking differentiator to obtain the tracking signal and the differential signal. The tracking differentiator is designed as follows:

$$\{ \begin{matrix}{f}_{h} = fhan ( {θ}_{r 1} - {θ}_{d} , {θ}_{r 2} , {r}_{0} , {h}_{0} ) \\ {θ}_{r 1} = {θ}_{r 2} \\ {{θ}^{`}}_{r 2} = {f}_{h}\end{matrix} \qquad ( 14 )$$

where fhan(θᵣ₁−θ_{d},θᵣ₂,r₀,h₀) is specifically expressed as follows:

$$( {θ}_{r 1} - {θ}_{d} , {θ}_{r 2} , {r}_{0} , {h}_{0} ) = - {r}_{0} ( \frac{{as}_{a}}{d} + sign ( a ) - {s}_{a} sign ( a ) ) \qquad ( 15 )$$

$$\{ \begin{matrix}d = {r}_{0} {h}_{0}^{2} , {a}_{0} = {h}_{0} {θ}_{r 2} , y = {θ}_{r 1} - {θ}_{d} + {a}_{0} \\ {a}_{1} = \sqrt{{d}^{2} + 8 d \vert y \vert} \\ {a}_{2} = {a}_{0} + sign ( y ) \frac{{a}_{1} - d}{2} \\ {s}_{y} = \frac{sign ( y + d ) - sign ( y - d )}{2} \\ a = {a}_{2} + {a}_{0} {s}_{y} + {ys}_{y} - {a}_{2} {s}_{y} \\ {s}_{a} = \frac{sign ( a + d ) - sign ( a - d )}{2}\end{matrix}$$

where r₀ represents a velocity factor of the tracking differentiator, when r₀ becomes larger, a tracking velocity of the tracking differentiator becomes faster, and the velocity factor is determined by the speed requirement for a transition process and the limit capabilities of the ADRBSC system; h₀ represents a filtering factor of the tracking differentiator, θ_{d} represents the target angle signal input into the tracking differentiator, θᵣ₁ represents the tracking signal, and θᵣ₂ represents the differential signal of θᵣ₁.

The tracking differentiator quickly tracks changes in the target angle signal by adjusting the velocity factor, enhancing dynamic response capabilities. Through the optimized design of the filtering factor, it effectively filters out high-frequency noise to generate the smooth signal, avoiding instability caused by the signal fluctuation. The tracking differentiator provides high-quality inputs for the extended state observer and the nonlinear error feedback controller, ensuring the precision of subsequent control and achieving high-precision control of the wrist joint angle. At the same time, through reasonable parameter design, the tracking differentiator adapts to different target angle change requirements, enhancing the robustness of the system to external disturbances and uncertainties.

The extended state observer is configured to estimate the state variables and the total system disturbance fₛ, and the total system disturbance fₛ is continuously differentiable and bounded and treated as an extended state

$${θ}_{f 3}^{r} .$$

A derivative of

$${θ}_{f 3}^{r} is {h}_{f} , i . e . , {\dot{θ}}_{f 3}^{r} = {h}_{f 3}^{r} , {h}_{f}$$

is essentially bounded, and thus the formula (12) is rewritten as follows:

$$\{ \begin{matrix}{\dot{θ}}_{f 1}^{r} = {θ}_{f 2}^{r} \\ {\dot{θ}}_{f 2}^{r} = {θ}_{f 3}^{r} + {b}_{0} u \\ {\dot{θ}}_{f 3}^{r} = {h}_{f}\end{matrix} \qquad ( 16 )$$

The third-order extended state observer is expressed as follows:

$$\{ \begin{matrix}{\tilde{e}}_{1} = {θ}_{f 1}^{g} - {θ}_{f 1}^{r} \\ {{θ}^{`}}_{f 1}^{g} = {θ}_{f 2}^{g} - {β}_{01} {ε}_{1} \\ {{θ}^{`}}_{f 2}^{g} = {θ}_{f 3}^{9} - {β}_{02} fal ( {ε}_{1} , {a}_{1} , δ ) + {b}_{0} u \\ {{θ}^{`}}_{f 3}^{g} = - {β}_{03} fal ( {ε}_{1} , {a}_{1} , δ )\end{matrix} \qquad ( 17 )$$

where \tilde{e}₁ represents an error between an angle observed value

$${θ}_{f 1}^{g}$$

and an output angle

$${θ}_{f 1}^{r} , {θ}_{f 1}^{g}$$

represents an estimated value of

$${θ}_{f 1}^{r} , {θ}_{f 2}^{g}$$

represents an estimated value of

$${θ}_{f 2}^{r} , {θ}_{f 3}^{g}$$

represents an estimated value of

$${θ}_{f 3}^{r} , {θ}_{f 3}^{r}$$

represents the extended state, and β₀₁, β₀₂ and β₀₃ represent gain coefficients of the extended state observer; and a function ƒal(\tilde{e}₁,aᵢ,δ) is defined as follows:

$$fal ( {\tilde{e}}_{i} , {a}_{i} , {δ}_{1} ) = \{ \begin{matrix}\frac{{\tilde{e}}_{i}}{{δ}^{1 - {a}_{i}}} , & \vert {\tilde{e}}_{i} \vert ⩽ {δ}_{1} \\ {\vert {\tilde{e}}_{i} \vert}^{{a}_{i}} sign ( {\tilde{e}}_{i} ) , & \vert {\tilde{e}}_{i} \vert > {δ}_{1}\end{matrix} \qquad ( 18 )$$

$$where {\tilde{e}}_{i} = {θ}_{fi}^{g} - {θ}_{fi}^{r} , i = 1 , 2 , 3 ;$$

δ₁ represents a linear interval length, and aᵢ represents a feedback exponent.

The extended state observer utilizes the error between the angle observed value and the output angle, and can rapidly and accurately estimate and feedback the system state and disturbance to the controller for compensation through the design of gain coefficients and the nonlinear function ƒal(\tilde{e}ᵢ,aᵢ,δ₁). The extended state observer can effectively overcome the influences of the unknown nonlinear term in the system and the external disturbance, significantly enhancing the robustness and the interference rejection capability of the system. By estimating the state variable and the total disturbance in real time, the extended state observer provides high-precision state information for subsequent controllers, ensuring the dynamic performance and stability of the ADRBSC system. At the same time, the nonlinear design of the extended state observer strengthens its adaptability to the complex dynamic system, providing important support for achieving high-precision trajectory tracking of the wrist joint.

In the disclosure, to accelerate the response speed, the nonlinear feedback controller adopts a non-smooth feedback approach, and uses the tracking signal θᵣ₁ and the differential signal θᵣ₂ generated by the tracking differentiator, and the estimated values

$${θ}_{f 1}^{g} and {θ}_{f 2}^{g}$$

from the extended state observer to form errors and generate an initial torque τ₁. The specific formula is expressed as follows:

$$\{ \begin{matrix}{e}_{1} = {θ}_{r 1} - {θ}_{f 1}^{g} \\ {e}_{2} = {θ}_{r 2} - {θ}_{f 2}^{g} \\ {τ}_{1} = {β}_{1} fal ( {e}_{1} , {a}_{i} , {δ}_{1} ) + {β}_{2} fal ( {e}_{2} , {a}_{i} , {δ}_{1} )\end{matrix} \qquad ( 19 )$$

By selecting appropriate controller parameters, it ensures that

$${θ}_{f 1}^{g} \to {θ}_{f 1}^{r} , {θ}_{f 2}^{g} \to {θ}_{f 2}^{r} ,$$

thereby obtaining the observed value

$${θ}_{f 3}^{g}$$

of the state vector

$${θ}_{f 3}^{r}$$

of the extended state observer. To eliminate the influence of the total disturbance

$${θ}_{f 3}^{g}$$

on the system, its estimated value

$${θ}_{f 3}^{g}$$

is compensated into the control input to obtain the final torque r required for the rotation of the pneumatic muscle wrist joint by θ_{d} as follows:

$$τ = \frac{{τ}_{1} - {θ}_{f 3}^{g}}{{b}_{0}} \qquad ( 20 )$$

The nonlinear feedback controller accelerates the system response speed through a non-smooth feedback design, ensuring the real-time performance of the ADRBSC system. The final control torque generated by the nonlinear feedback controller can precisely drive the PAM wrist joint system, achieving high-precision trajectory tracking control of the wrist joint movement, thereby enhancing the dynamic performance and stability of the ADRBSC system.

In the disclosure, the backstepping controller is configured to perform the following steps:
A1, based on the control torque, using an inverse operation of the dynamic model of the single-joint mechanical arm to calculate the output pull force of the first PAM system and the output pull force of the second PAM system when the PAM wrist joint system rotates by an angle Bd; and
A2, substituting the output pull force of the first PAM system and the output pull force of the second PAM system into the PAM system mathematical model to calculate the output pressure of the first PAM system and the output pressure of the second PAM system.

Specifically, according to the backstepping principle, the control torque r is generated by the active disturbance rejection control algorithm. By an inversely operation of the dynamic model of the single-joint robotic arm, i.e., the formula (10), the output pull forces of the first PAM system and the second PAM system for the rotation of the PAM wrist joint system by the angle θ_{d} are calculated. The specific formulas are as follows:

$$\{ \begin{matrix}{F}_{1} ( {ε}_{1} , {p}_{q 1} ) = \frac{τ}{r sin {γ}_{1}} , {F}_{2} ( {ε}_{2} , {p}_{q 2} ) & τ > 0 \\ {F}_{1} ( {ε}_{1} , {p}_{q 1} ) = 0 , {F}_{2} ( {ε}_{2} , {p}_{q 2} ) = \frac{τ}{r sin {γ}_{2}} & τ < 0 ( 21 ) \\ {F}_{1} ( {ε}_{1} , {p}_{q 1} ) = 0 , {F}_{2} ( {ε}_{2} , {p}_{q 2} ) = 0 & τ = 0\end{matrix} \qquad ( 21 )$$

where F₁(ε₁,p_{q1}) represents the output pull force of the first PAM system, F₂(ε₂,p_{q2}) represents the output pull force of the second PAM system, and τ represents the control torque.

The formula (21) is substituted into the formula (8) for the PAM system mathematical model, after rearranging, to calculate the output pressure of the first PAM system and the output pressure of the second PAM system. The specific formulas are expressed as follows:

$$\{ \begin{matrix}{p}_{r 1} = \frac{{F}_{1} ( {ε}_{1} , {p}_{q 1} ) - h ( {ε}_{1} )}{g ( {ε}_{1} )} \\ {p}_{r 2} = \frac{{F}_{2} ( {ε}_{2} , {p}_{q 2} ) - h ( {ε}_{2} )}{g ( {ε}_{2} )}\end{matrix} \qquad ( 22 )$$

where pᵣ₁ represents the output pressure of the first PAM system, and pᵣ₂ represents the output pressure of the second PAM system.

Due to the hysteresis effect of the proportional pressure valves, the relationship between the control rate u and the output pressure P is variable. Utilizing the model-free characteristic of the PID controllers, the pressure commands required for the first PAM system and the second PAM system, obtained from the backstepping principle, can be processed through PID control to overcome these adverse factors, which allows for the correction of the control rates of the first proportional pressure valve and the second proportional pressure valve, thus obtaining the final control rates u₀, u₁. Consequently, the precise control of the pressure commands for the first PAM system and the second PAM system is thereby achieved, completing the accurate trajectory tracking control of the entire ADRBSC system.

The backstepping controller, by integrating the active disturbance rejection control algorithm with the inverse operation of the single-joint mechanical arm model, precisely calculates the required output pull force and corresponding output pressure for the PAM wrist joint system. The backstepping controller also combines the model-free characteristics of the PID controllers to correct the hysteresis nonlinearity of the proportional pressure valves, ensuring precise control of the pressure commands for the first PAM system and the second PAM system. This design effectively overcomes hysteresis, nonlinearity, and deviation issues within the PAM systems, achieving high-precision control of the wrist joint rotation angle.

Through the hierarchical recursive control of the backstepping method, the system significantly enhances the trajectory tracking performance and the dynamic response capabilities, ensuring the accuracy and robustness in the control process, and providing reliable assurance for the accurate and safe execution of wrist joint movements during rehabilitation training.

Specifically, the ADRBSC system further includes: a sine wave function generation module and a minimum acceleration model generation module.

The sine wave function generation module is configured to generate the target angle signal as follows:

$${θ}_{d} = A sin ( π ft - 0.5 π ) + B \qquad ( 23 )$$

where θ_{d} represents the target angle signal, A represents a sine wave amplitude, f represents a sine wave frequency, and B represents a sine wave angle bias;
The minimum acceleration model generation module is configured to generate an optimal wrist motion curve. The minimum acceleration model meets the following mathematical law:

$${θ}_{f} ( {t}_{0} ) = {θ}_{0} , {θ}_{f} ( {t}_{1} ) = {θ}_{1} \qquad ( 24 )$$

$${min}_{{θ}_{f}} I = \frac{1}{2} {\int }_{{t}_{0}}^{{t}_{1}} {{θ}^{...}}_{f}^{2} dt$$

where t₀ represents a starting moment of the joint movement, θ₀ represents an original position of a joint movement, t₁ represents a stopping moment of the joint movement, θ₁ represents a stopping position of the joint movement, and \ddot{θ}_{f} represents a joint angular acceleration, which is the third derivative of the joint angle with respect to time.

By combining the minimum acceleration model and applying the calculus of variations (also referred to as variation method), the optimal joint angle curve is obtained as follows:

$${θ}_{d} = {θ}_{0} + ( {θ}_{1} - {θ}_{0} ) ( \frac{10 {t}^{3}}{{d}^{3}} - \frac{15 {l}^{4}}{{d}^{4}} + \frac{6 {t}^{5}}{{d}^{5}} ) \qquad ( 25 )$$

$$d = {t}_{1} - {t}_{0} , t ∈ [ {t}_{0} , {t}_{1} ]$$

Respectively maintaining a certain delay time of ¼T at the lowest and highest positions, the optimal wrist motion control curve is finally obtained as follows:

$${θ}_{d} = {θ}_{0} + ( {θ}_{1} - {θ}_{0} ) ( \frac{10 {t}^{3}}{{d}^{3}} - \frac{15 {l}^{4}}{{d}^{4}} + \frac{6 {t}^{5}}{{d}^{5}} ) , d = {τ}_{1} - {τ}_{0} \qquad ( 26 )$$

$${θ}_{0} = 10 , {θ}_{1} = 10 when t ∈ [ nT , 1 / 4 T + nT )$$

$${θ}_{0} = 10 , {θ}_{1} = 50 when t ∈ [ 1 / 4 T + T , 1 / 2 T + nT )$$

$${θ}_{0} = 50 , {θ}_{1} = 50 when t ∈ [ 1 / 2 T + T , 3 / 4 T + nT )$$

$${θ}_{0} = 50 , {θ}_{1} = 10 when t ∈ [ nT , 1 / 4 T + nT )$$

where θ₀ represents the original position of a joint movement, θ₁ represents the stopping position of the joint movement, T represents a period of an optimal wrist motion control curve, n represents a number of repetitions of a wrist motion control curve, t represents a time value, d represents an interval time, t₁ represents the stopping moment of the joint movement, and t₀ represents the starting moment of the joint movement

Specifically, in an embodiment, the ADRBSC system is configured to perform the following steps S1-S7:
S1, the tracking differentiator receives the target angle signal, and generates the tracking signal which is smooth and the differential signal of the tracking signal.

Specifically, the tracking differentiator uses a filtering algorithm (such as a gradient descent method or a sliding mode control method) to eliminate high-frequency noise from the input signal, ensuring that the generated signal has good continuity and differentiability.
S2, the extended state observer estimates the system state and the total system disturbance according to a system input and a system output.

Specifically, by utilizing the extended state observer to treat the unknown disturbance as an extended state and performing real-time estimation through an observer algorithm. The introduction of the extended state observer significantly enhances the interference rejection capability of the ADRBSC system, allowing the controller to maintain high-precision control in complex nonlinear environments.
S3, the nonlinear error feedback controller generates the control torque based on state estimation and disturbance estimation.

Specifically, the nonlinear error feedback controller generates an error feedback signal by combining the tracking signal, the differential signal, and the estimated state value, and further calculates the control torque required for the system. The working principle of the nonlinear error feedback controller is based on the nonlinear control theory, where a nonlinear error feedback function is designed to nonlinearly amplify or suppress the error, thereby improving the dynamic performance and steady-state accuracy of the ADRBSC system.
S4, the backstepping controller allocates the output pressure of the first PAM system and the output pressure of the second PAM system according to system dynamics and a PAM system mathematical model.

Specifically, the backstepping controller first calculates the output pull force of the PAM system module based on the control torque, and then substitutes the output pull force of the PAM system module into the PAM system mathematical model to further calculate the required output pressure. The backstepping control strategy, through a step-by-step recursive approach, decomposes complex nonlinear control problems into multiple sub-problems, which are solved step by step, thereby achieving precise control of the nonlinear system.
S5, the PID controllers track and control the output pressure of the first PAM system and the output pressure of the second PAM system.

Specifically, due to the nonlinear hysteresis relationship between the output pressure and the control rate of the proportional pressure valve, the PID controllers utilizes the model-free characteristics to dynamically compensate for pressure errors by real-time adjustment of the proportional, integral, and derivative parameters. This ensures the accuracy and real-time performance of pressure control. The introduction of the PID controllers significantly enhances the robustness of the system, enabling the system to adapt to various complex operating conditions.
S6, the adaptive combination valve executes the control commands, and adjusts the pressure within the PAM system module.

Specifically, the adaptive combination valve includes the two proportional pressure valves, the control unit, and the pressure sensor. The primary function of the adaptive combination valve is to execute the control commands and regulate the gas pressure within the PAM system module. To be more specific, the adaptive combination valve dynamically adjusts the opening of the proportional pressure valves by real-time monitoring the pressure state of the PAM system module, thereby achieving precise control of the gas pressure.
S7, the encoder records a rotation angle of the wrist joint in real time, and feeds the rotation angle of the wrist joint back to the ADRBSC system to form a closed-loop control.

Specifically, the encoder provides real-time position information of the wrist joint through the high-precision angle measurement technology, supplying crucial feedback data to the ADRBSC system. The closed-loop control mechanism dynamically adjusts the control strategy using the feedback data, thereby achieving high-precision control of the wrist joint movement while ensuring the stability and safety of the system.

In an embodiment of the disclosure, experiments are conducted on the ADRBSC system to evaluate the performance of the ADRBSC system.

The first set of experiments involved sine wave trajectory tracking. The controller parameters for the ADRBSC system are set as follows:

| parameter | value | parameter | value |
|---|---|---|---|
| Li | 234 mm ≤ Lᵢ ≤ 313 mm | hₒ | 0.01 |
| r | 54.2 mm | h | 0.01 |
| L₀ | 273.15 mm | rₒ | 300 |
| L_{AC} | 256 | β₀₁ | 15 |
| θ_{d} | −45° ≤ θ_{w} ≤ 45° | β₀₂ | 65 |
| Lₒᵣᵢ | 292 | β₀₃ | 3375 |
| f_{1, 1} | 778 | β₁ | 150 |
| f_{2, 1} | 2423 | β₂ | 20 |
| f_{3, 1} | 24.16 | δ | 0.5 |
| f_{1, 2} | −121.3 | σ₁ | 1.2 |
| f_{2, 2} | 119.9 | σ₂ | 0.75 |
| f_{3, 2} | 130.5 | | |
| b₀ | 2000 | | |

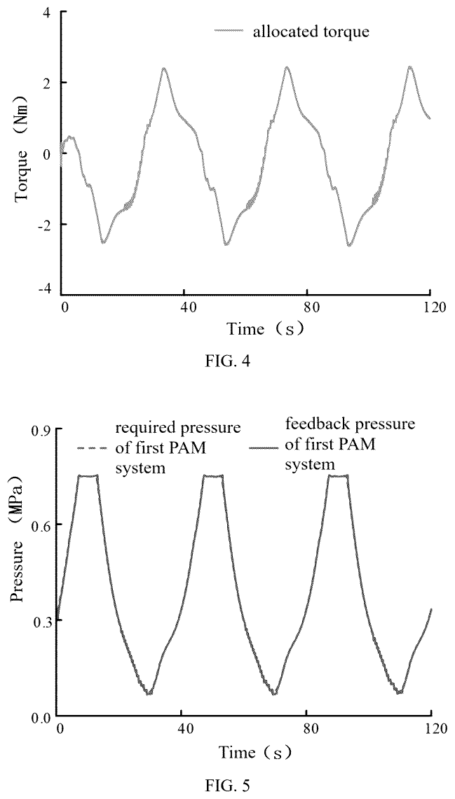

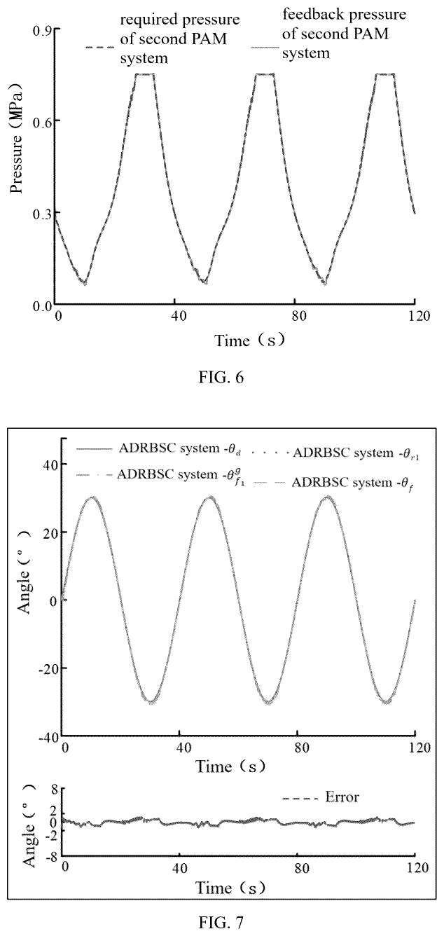

The results of the first set of experiments are shown in FIGS. 4-7. FIG. 4 illustrates the torque generated by the active disturbance rejection control algorithm for sine wave trajectory tracking control, and the torque is the optimal torque allocated by the active disturbance rejection control algorithm for the wrist joint rotation by θ_{d}. FIG. 5 shows the tracking control of the pressure allocated to the first PAM system by the backstepping method for the sine wave trajectory tracking control; where the dashed line represents the pressure required for the wrist joint rotation by θ_{d} of the first PAM system, allocated through the ADRBSC method, and the solid line represents the feedback pressure. FIG. 6 shows the tracking control of the pressure allocated to the second PAM system by the backstepping method for the sine wave trajectory tracking control; where the dashed line represents the pressure required for the wrist joint rotation by θ_{d} of the second PAM system, allocated through the ADRBSC method, and the solid line represents the feedback pressure. It can be seen from the figures that the control algorithm enables the feedback pressure to quickly track the required pressure for both the first PAM system and the second PAM system, with a high level of control precision, achieving good pressure tracking control performance. FIG. 7 shows the experimental results of the ADRBSC sine wave trajectory tracking control. It can be seen from the figure that the output θᵣ₁ of the tracking differentiator accurately tracks the given θ_{d}, and the observed value

$${θ}_{f 1}^{g}$$

of the rotation angle is almost consistent with Of. At the beginning of the trajectory, the feedback trajectory Of can quickly track the given trajectory θ_{d}, and throughout the subsequent trajectory tracking control, a high level of control precision is maintained, with the control error kept within 2°.

The second set of experiments includes optimal wrist curve trajectory tracking. In this experiment, the ADRBSC controller parameters are set the same as in the first set of experiments, with the difference being that the values of β₁ and β₂ are changed to 200 and 40, respectively.

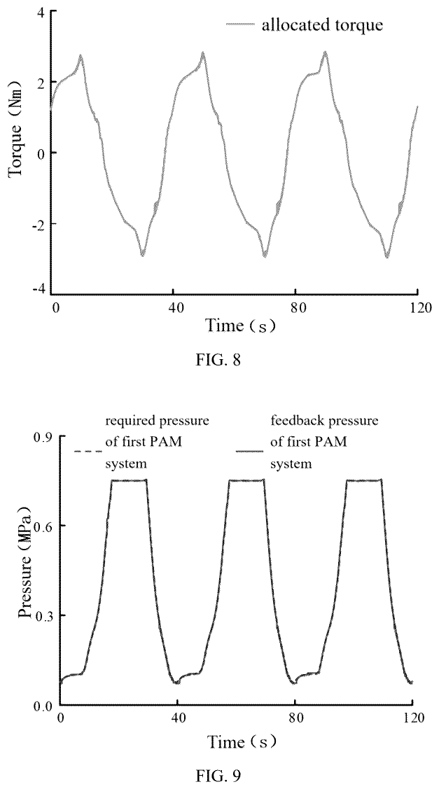

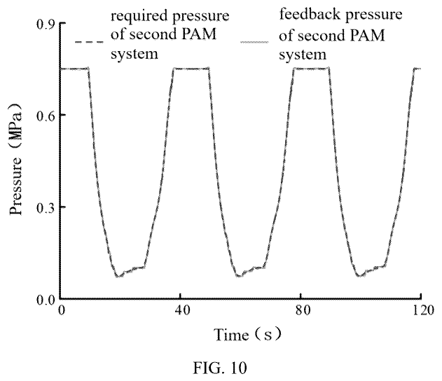

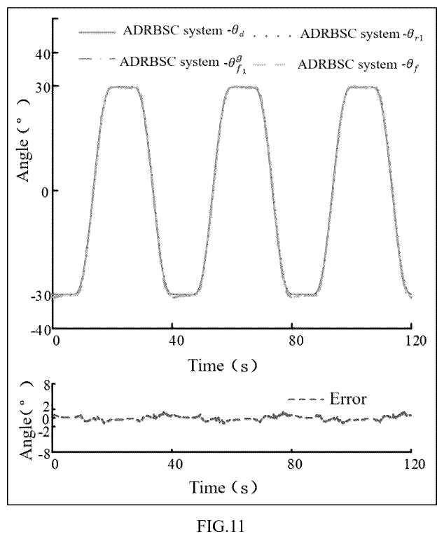

The results of the second set of experiments are shown in FIGS. 8-11. FIG. 8 illustrates the torque generated by the active disturbance rejection control algorithm for optimal wrist curve trajectory control. This torque is the optimal torque allocated by the active disturbance rejection control algorithm for the wrist joint rotation by θ_{d}. FIG. 9 shows the tracking control of the pressure allocated to the first PAM system by the backstepping method for optimal wrist curve trajectory tracking control. FIG. 10 shows the tracking control of the pressure allocated to the second PAM system by the backstepping method for the optimal wrist curve trajectory tracking control. The pressure tracking control results for the first PAM system and the second PAM system in this allocation are similar to those in the sine wave trajectory tracking control, maintaining a high level of pressure tracking control precision. FIG. 11 shows the experimental results of the ADRBSC optimal wrist curve trajectory tracking control. Both in terms of the initial trajectory response speed and the overall trajectory tracking control precision, the results for the optimal curve trajectory are consistent with those of the sine wave control, maintaining a good control effect.

The above description is only the illustrated embodiments of the disclosure and is not intended to limit the disclosure. Any amendments, equivalent substitutions, improvements, etc. made within the spirit and principles of the disclosure should be included in the scope of protection of the disclosure.

What is claimed is:

1. An active disturbance rejection backstepping control (ADRBSC) system for a wrist rehabilitation robot, wherein the wrist rehabilitation robot comprises two pneumatic artificial muscle (PAM) systems as driving components, the two PAM systems are a first PAM system and a second PAM system, and the first PAM system and the second PAM system form a PAM system module; and the ADRBSC system comprises:
a tracking differentiator, configured to track and differentiate a target angle signal input thereinto to generate a tracking signal and a differential signal;
an extended state observer, configured to estimate a system state and a total system disturbance in real time, and output an estimated state value;
a nonlinear error feedback controller, configured to form an error feedback signal and generate a control torque based on the tracking signal, the differential signal, and the estimated state value;
a backstepping controller, configured to calculate and allocate, based on the control torque and using a dynamic model of a single-joint mechanical arm, an output pressure of the first PAM system and an output pressure of the second PAM system;
proportion-integration-differentiation (PID) controllers, configured to dynamically correct the output pressure of the first PAM system and the output pressure of the second PAM system to compensate for a hysteresis effect of proportional pressure valves; and
an adaptive combination valve, comprising two proportional pressure valves, a control unit, and a pressure sensor, and configured to execute control commands and monitor a pressure within the PAM system module.

2. The ADRBSC system for the wrist rehabilitation robot as claimed in claim 1, wherein the dynamic model of the single-joint mechanical arm is as follows:
$$τ = J {\ddot{θ}}_{f} + {b}_{v} {\dot{θ}}_{f} + {f}_{1}$$
$$= [ {F}_{1} ( {ε}_{1} , {p}_{q 1} ) sin {γ}_{1} - {F}_{2} ( {ε}_{2} , {p}_{q 2} ) sin {γ}_{2} ] r$$
where τ represents the control torque, J represents a rotational inertia of a joint pulley, which is obtained from a formula of J=½mr², b. represents a joint damping coefficient, f₁ represents an unknown external disturbance, θ_{f} represents a rotation angle of the joint pulley, p_{q1} represents a chamber pressure of the first PAM system, p_{q2} represents a chamber pressure of the second PAM system, r represents a radius of the joint pulley, F₁(ε₁, p_{q1}) represents an output pull force of the first PAM system, F₂(ε₂,p_{q2}) represents an output pull force of the second PAM system, E represents a contraction ratio of the PAM system module, ε=1−L/Lₒᵣᵢ, Lₒᵣᵢ represents an initial length of the PAM system module, and L represents an actual length of the PAM system module; and
b₀=1/J, −bᵥ/J\dot{θ}_{f}+f₁/J is the total system disturbance fₛ,
$${θ}_{f 1}^{r} = {θ}_{f} , {θ}_{f 2}^{r} = {\dot{θ}}_{f} ,$$
and thus a system state space formula is expressed as follows:
$$\{ \begin{matrix}{\dot{θ}}_{f 1}^{r} = {θ}_{f 2}^{r} \\ {\dot{θ}}_{f 2}^{r} = {f}_{s} + {b}_{0} τ\end{matrix}$$
where fₛ represents the total system disturbance, comprising the unknown external disturbance.

3. The ADRBSC system for the wrist rehabilitation robot as claimed in claim 1, wherein the tracking differentiator is designed as follows:
$$\{ \begin{matrix}{f}_{h} = fhan ( {θ}_{r 1} - {θ}_{d} , {θ}_{r 2} , {r}_{0} , {h}_{0} ) \\ {\dot{θ}}_{r 1} = {θ}_{r 2} \\ {\dot{θ}}_{r 2} = {f}_{h}\end{matrix}$$
where fhan(θᵣ₁−θ_{d}, θᵣ₂, r₀, h₀) is specifically expressed as follows:
$$( {θ}_{r 1} - {θ}_{d} , {θ}_{r 2} , {r}_{0} , {h}_{0} ) = - {r}_{0} ( \frac{{as}_{a}}{d} + sign ( a ) - {s}_{a} sign ( a ) )$$
$$\{ \begin{matrix}d = {r}_{0} {h}_{0}^{2} , {a}_{0} = {h}_{0} {θ}_{r 2} , y = {θ}_{r 1} - {θ}_{d} + {a}_{0} \\ {a}_{1} = \sqrt{{d}^{2} + 8 d \vert y \vert} \\ {a}_{2} = {a}_{0} + sign ( y ) \frac{{a}_{1} - d}{2} \\ {s}_{y} = \frac{sign ( y + d ) - sign ( y - d )}{2} \\ a = {a}_{2} + {a}_{0} {s}_{y} + {ys}_{y} - {a}_{2} {s}_{y} \\ {s}_{a} = \frac{sign ( a + d ) - sign ( a - d )}{2}\end{matrix}$$
where r₀ represents a velocity factor of the tracking differentiator, when r₀ is larger, a tracking velocity of the tracking differentiator is faster; h₀ represents a filtering factor of the tracking differentiator, θ_{d} represents the target angle signal input into the tracking differentiator, θᵣ₁ represents the tracking signal, and θᵣ₂ represents the differential signal of θᵣ₁.

4. The ADRBSC system for the wrist rehabilitation robot as claimed in claim 2, wherein an unknown nonlinear term f(θ_{f}, \dot{θ}_{f}) of the ADRBSC system is a state variable of the extended state observer, a derivative of f(θ_{f}, \dot{θ}_{f}) is h(θ_{f}, \dot{θ}_{f}), h(θ_{f}, \dot{θ}_{f})=f(θ_{f}, \dot{θ}_{f}), the extended state observer of three stages is designed as follows:
$$\{ \begin{matrix}{\tilde{e}}_{1} = {θ}_{f 1}^{g} - {θ}_{f 1}^{r} \\ {\dot{θ}}_{f 1}^{g} = {θ}_{f 2}^{g} - {β}_{01} {\tilde{e}}_{1} \\ {\dot{θ}}_{f 2}^{g} = {θ}_{f 3}^{g} - {β}_{02} fal ( {\tilde{e}}_{1} , {a}_{1} , δ ) + {b}_{0} u \\ {\dot{θ}}_{f 3}^{g} = - {β}_{03} fal ( {\tilde{e}}_{1} , {a}_{1} , δ )\end{matrix}$$
where \tilde{e}₁ represents an error between an angle observed value
$${θ}_{f 1}^{g}$$
and an out angle
$${θ}_{f 1}^{r} , {θ}_{f 1}^{g}$$
represents an estimated value of
$${θ}_{f 1}^{r} , {θ}_{f 2}^{g}$$
represents an estimated value of
$${θ}_{f 2}^{r} , {θ}_{f 3}^{g}$$
represents an estimated value of
$${θ}_{f 3}^{r} , {θ}_{f 3}^{r}$$
represents an extended state, and β₀₁, β₀₂ and β₀₃ represent gain coefficients of the extended state observer; and a function ƒal(εᵢ, aᵢ, δ) is defined as follows:
$$fal ( {\tilde{e}}_{i} , {a}_{i} , δ ) = \{ \begin{matrix}\frac{{\tilde{e}}_{i}}{{δ}^{1 - {a}_{i}}} , & \vert {\tilde{e}}_{i} \vert ⩽ δ \\ {\vert {\tilde{e}}_{i} \vert}^{{a}_{i}} sign ( {\tilde{e}}_{i} ) , & \vert {\tilde{e}}_{i} \vert > δ\end{matrix}$$
$$where {\tilde{e}}_{i} = {θ}_{fi}^{g} - {θ}_{fi}^{r} , i = 1 , 2 , 3 ;$$
δ represents a linear interval length, and aᵢ represents a feedback exponent.

5. The ADRBSC system for the wrist rehabilitation robot as claimed in claim 4, wherein the nonlinear error feedback controller is designed as follows:
$$\{ \begin{matrix}{e}_{1} = {θ}_{r 1} - {θ}_{f 1}^{g} \\ {e}_{2} = {θ}_{r 2} - {θ}_{f 2}^{g} \\ τ = \frac{{β}_{1} fal ( {e}_{1} , {a}_{i} , {δ}_{1} ) + {β}_{2} fal ( {e}_{2} , {a}_{i} , {δ}_{1} ) - {θ}_{f 3}^{g}}{{b}_{0}}\end{matrix}$$
where e₁ represents an error between a given angle θᵣ₁ and an estimated angle value
$${θ}_{f 1}^{g} ,$$
and e₂ represents an error between a given angle θᵣ₂ and an estimated angle value
$${θ}_{f 2}^{g} .$$

6. The ADRBSC system for the wrist rehabilitation robot as claimed in claim 1, wherein the backstepping controller is configured to perform the following steps:
A1, based on the control torque, using an inverse operation of the dynamic model of the single-joint mechanical arm to calculate an output pull force of the first PAM system and an output pull force of the second PAM system when a wrist joint system of the PAM system module rotates by an angle θ_{d}; and
A2, substituting the output pull force of the first PAM system and the output pull force of the second PAM system into a PAM system mathematical model to calculate the output pressure of the first PAM system and the output pressure of the second PAM system.

7. The ADRBSC system for the wrist rehabilitation robot as claimed in claim 6, wherein the PAM system mathematical model is expressed as follows:
$$\{ \begin{matrix}F ( ε , {p}_{q} ) = g ( ε ) {p}_{q} + h ( ε ) \\ g ( ε ) = 778 - 2423 ε + 24.16 exp ( - 38.74 ε ) \\ h ( ε ) = - 121.3 - 119.9 ε + 130.5 exp ( - 38.74 ε )\end{matrix}$$
where F(ε, p_{q}) represents the output pull force of the PAM system module, ε represents a contraction ratio of the PAM system module, and p_{q} represents a chamber pressure of the PAM system module.

8. The ADRBSC system for the wrist rehabilitation robot as claimed in claim 7, wherein calculation formulas of the output pull force of the first PAM system and the output pull force of the second PAM system are as follows:
$$\{ \begin{matrix}{F}_{1} ( {ε}_{1} , {p}_{q 1} ) = \frac{τ}{r sin {γ}_{1}} , {F}_{2} ( {ε}_{2} , {p}_{q 2} ) = 0 & τ > 0 \\ {F}_{1} ( {ε}_{1} , {p}_{q 1} ) = 0 , {F}_{2} ( {ε}_{2} , {p}_{q 2} ) = \frac{τ}{r sin {γ}_{2}} & τ < 0 \\ {F}_{1} ( {ε}_{1} , {p}_{q 1} ) = 0 , {F}_{2} ( {ε}_{2} , {p}_{q 2} ) = 0 & τ = 0\end{matrix}$$
where F₁(ε₁, p_{q1}) represents the output pull force of the first PAM system, F₂(ε₂, p_{q2}) represents the output pull force of the second PAM system, r represents the control torque, and r represents a radius of a joint pulley;
wherein calculation formulas of the output pressure of the first PAM system and the output pressure of the second PAM system are as follows:
$$\{ \begin{matrix}{p}_{r 1} = \frac{{F}_{1} ( {ε}_{1} , {p}_{q 1} ) - h ( {ε}_{1} )}{g ( {ε}_{1} )} \\ {p}_{r 2} = \frac{{F}_{2} ( {ε}_{2} , {p}_{q 2} ) - h ( {ε}_{2} )}{g ( {ε}_{2} )}\end{matrix}$$
where pᵣ₁ represents the output pressure of the first PAM system, and pᵣ₂ represents the output pressure of the second PAM system.

9. The ADRBSC system for the wrist rehabilitation robot as claimed in claim 1, further comprising:
a sine wave function generation module, configured to generate the target angle signal as follows:
$${θ}_{d} = A sin ( πft - 0.5 π ) + B$$
where θ_{d} represents the target angle signal, A represents a sine wave amplitude, f represents a sine wave frequency, and B represents a sine wave angle bias; and
a minimum acceleration model generation module, configured to generate an optimal wrist motion curve as follows:
$${θ}_{d} = {θ}_{0} + ( {θ}_{1} - {θ}_{0} ) ( \frac{10 {t}^{3}}{{d}^{3}} - \frac{15 {t}^{4}}{{d}^{4}} + \frac{6 {t}^{5}}{{d}^{5}} ) , d = {t}_{1} - {t}_{0}$$
$${θ}_{0} = 10 , {θ}_{1} = 10 when t ∈ [ nT , 1 / 4 T + nT )$$
$${θ}_{0} = 10 , {θ}_{1} = 50 when t ∈ [ 1 / 4 T + T , 1 / 2 T + nT )$$
$${θ}_{0} = 50 , {θ}_{1} = 50 when t ∈ [ 1 / 2 T + T , 3 / 4 T + nT )$$
$${θ}_{0} = 50 , {θ}_{1} = 10 when t ∈ [ nT , 1 / 4 T + nT )$$
where θ₀ represents an original position of a joint movement, θ₁ represents a stopping position of the joint movement, T represents a period of an optimal wrist motion control curve, n represents a number of repetitions of a wrist motion control curve, t represents a time value, d represents an interval time, t₁ represents a stopping moment of the joint movement, and t₀ represents a starting moment of the joint movement.

10. The ADRBSC system for the wrist rehabilitation robot as claimed in claim 1, configured to perform the following steps:
S1, receiving, by the tracking differentiator, the target angle signal, and generating, by the tracking differentiator, the tracking signal which is smooth and the differential signal of the tracking signal;
S2, estimating, by the extended state observer, the system state and the total system disturbance according to a system input and a system output;
S3, generating, by the nonlinear error feedback controller, the control torque based on the system state and the total system disturbance;
S4, allocating, by the backstepping controller, the output pressure of the first PAM system and the output pressure of the second PAM system according to system dynamics and a PAM system mathematical model;
S5, tracking and controlling, by the PID controllers, the output pressure of the first PAM system and the output pressure of the second PAM system;
S6, executing, by the adaptive combination valve, the control commands, and adjusting, by the adaptive combination valve, the pressure within the PAM system module; and
S7, recording, by an encoder, a rotation angle of a wrist joint in real time, and feeding, by the encoder, the rotation angle of the wrist joint back to the ADRBSC system to form a closed-loop control.
